(19) United States
(12) Patent Application Publication
(10) Pub. No.: US 2012/0045821 A1
(43) Pub. Date: Feb. 23, 2012

(54) METHODS AND COMPOSITIONS FOR GENETIC ENGINEERING OF CYANOBACTERIA TO PRODUCE ETHANOL

(76) Inventor: Pengcheng Fu, Honolulu, HI (US)

(21) Appl. No.: 13/148,469

(22) PCT Filed: Feb. 12, 2009
(86) PCT No.: PCT/US09/33959

§ 371 Date: Nov. 10, 2011

(51) Int. Cl. C12N 1/13; C12N 15/74

(52) U.S. Cl. 435/257.2; 435/320.1

(57) ABSTRACT

Provided herein are compositions and methods for genetic engineering of cyanobacteria to produce ethanol. In one aspect, the present invention provides a polynucleotide construct comprising a copper ion inductive promoter and a sequence encoding a pyruvate decarboxylase (pdc) enzyme. In another aspect, the present invention provides a genetically engineered cyanobacterium comprising the polynucleotide construct of the invention, wherein the cyanobacterium is capable of producing ethanol after a period of fermentation. In yet another aspect, the present invention discloses a method of producing ethanol by genetically modifying cyanobacteria using the polynucleotide construct of the invention.

### BACKGROUND OF THE INVENTION

Development of renewable energy is rapidly embraced by society and industry to meet energy growth and emission reduction goals. Since the industrial revolution, the world's economy has relied heavily on fossil fuels as energy sources. Reliance on these energy sources has created several challenging problems, such as reduced supply of fossil fuel resources, environmental pollution and the consequent global warming effect. One alternative to fossil fuels is ethanol. The current world ethanol production is 60% from sugar crops, 33% from other crops and 7% from chemical synthesis. Traditional biomass ethanol production processes require vast quantities of arable land and energy input requirement for the growth of the feedstock. Furthermore, traditional fermentation methods release considerable quantities of CO₂ as a byproduct of the fermentation process. For example, a 40 MMGY (million gallons per year) biomass ethanol plant may release 121,000 tons of CO₂ each year into the environment (BBI, 2003). This greenhouse gas will worsen the global warming effect.

Bioethanol has recently surged to the forefront of renewable fuels technology. It provides a viable alternative to petroleum based fuels, offering control over both production and consumption processes. In addition, ethanol derived from biological systems is particularly attractive because it can be readily integrated into numerous existing infrastructures; considering both production and fuel industries. Various methods for ethanol production by living organisms have been investigated. The production of ethanol by microorganisms has, in large part, been investigated using the yeast Saccharomyces cerevisiae and the obligately ethanogenic bacteria Zymomonas mobilis. Both of these microorganisms contain the genetic information to produce the enzymes pyruvate decarboxylase (pdc) and alcohol dehydrogenase (adh), which are used to produce ethanol from pyruvate, a product of the glycolytic pathway. Woods et al. (U.S. Pat. Nos. 6,306,639 and 6,699,696; see also Deng and Coleman, “Ethanol Synthesis by Genetic Engineering in Cyanobacteria” Applied and Environmental Microbiology (1999) 65(2):523-428) disclose a genetically modified cyanobacterium useful for the production of ethanol. Woods et al. report an ethanol production level of 5 mM after 30 days of culture. It is therefore desirable to find a simple, efficient and cost-effective biological system for producing substantial amounts of ethanol.

### SUMMARY OF THE INVENTION

In one aspect, the present invention provides a polynucleotide construct comprising: a copper ion inducible promoter and a polynucleotide sequence encoding a pyruvate decarboxylase (pdc) enzyme. In some embodiments, the copper ion inductive promoter is pPetE promoter. In some embodiments, the polynucleotide sequence encoding pdc enzyme is obtained from Acetobacter pasteurianus plasmid pGADL201. In some embodiments, the polynucleotide sequence encoding pdc enzyme is obtained from Gluconobacter suboxydans. In some embodiments, the polynucleotide sequence encoding pdc enzyme comprises SEQ. ID NO: 3 or a pdc enzyme-encoding polynucleotide sequence that is capable of being expressed in cyanobacteria. In some embodiments, the sequence encoding pdc enzyme comprises a nucleic acid sequence encoding an amino acid sequence of SEQ ID NO: 8. The present invention also discloses an expression vector comprising a polynucleotide construct, which comprises a copper ion inducible promoter and a polynucleotide sequence encoding a pyruvate decarboxylase (pdc) enzyme. Also provided by the present invention is a host cell comprising the expression vector disclosed herein. In some embodiments, the expression vector is integrated into the host cell chromosome. In some embodiments, the expression vector is pPETPDC. In some embodiments, the cell is a cyanobacterium. In some embodiments, the cyanobacterium is Synechocystis. In some embodiments, the cyanobacterium is Synechocystis sp. PCC 6803, or other transformable strain of Synechocystis. In some embodiments, the cyanobacterium is a wild-type strain of Synechocystis sp. PCC 6803. In some embodiments, the cyanobacterium is Synechococcus PCC 7942, or other transformable strain of Synechococcus. In some embodiments, the host cell produces ethanol in a quantifiable amount after a period of copper ion induction. In some embodiments, the host cell produces ethanol in a quantity that is greater than about 50 mM ethanol after about 8 days of fermentation.

In another aspect, the present invention provides a genetically engineered cyanobacterium comprising a polynucleotide construct, which comprises a polynucleotide sequence encoding pyruvate decarboxylase (pdc) enzyme and a copper ion inducible promoter, wherein the cyanobacterium is capable of producing ethanol. In some embodiments, the ethanol is produced in a quantity that is greater than about 50 mM after about 8 days of fermentation. In some embodiments, the cyanobacterium is resistant to high temperature and high ethanol concentration. In some embodiments, the cyanobacterium is derived from directed evolution by heat shock to increase cellular tolerance to high temperature and high ethanol concentration. In some embodiments, the polynucleotide sequence encoding pdc enzyme is obtained from Acetobacter pasteurianus plasmid pGADL201. In some embodiments, the polynucleotide sequence encoding pdc enzyme is obtained from Gluconobacter suboxydans. In some embodiments, the cyanobacterium is Synechocystis including the strain Synechocystis sp. PCC 6803, or other transformable strains of Synechocystis. In some embodiments, the cyanobacterium is a wild type Synechocystis sp. PCC 6803 strain. In some embodiments, the polynucleotide sequence encoding the pdc enzyme comprises SEQ. ID NO: 3 that is capable of being expressed in cyanobacteria. In some embodiments, the polynucleotide sequence is a sequence encoding the pdc enzyme comprising SEQ. ID NO: 8. In some embodiments, the copper ion inducible promoter is a pPetE promoter. In some embodiments, the polynucleotide construct is pPETPDC.

In yet another aspect, the present invention discloses a method for producing ethanol comprising: (a) creating a cyanobacteria mutant that is resistant to high temperature and high ethanol concentration by heat-shock related directed evolution; (b) genetically modifying the cyanobacteria mutant by introducing a construct comprising a polynucleotide sequence encoding pdc enzyme, and a copper ion responsive promoter; (c) adding copper to the genetically modified cyanobacteria mutant to induce ethanol production; and (d) collecting ethanol after a period of fermentation. In some embodiments, the cyanobacteria produce ethanol in a recoverable quantity that is about 50 mM ethanol after about 8 days of fermentation. In some embodiments, the cyanobacteria produce ethanol in a recoverable quantity that is between about 20 mM to about 100 mM ethanol after about 8 days of fermentation. In some embodiments, the ethanol concentration of the culture medium is at least about 20 mM, 30 mM, 40 mM, 50 mM, or 60 mM after about eight days of culture or fermentation. In some embodiments, the ethanol concentration of the culture medium is at least about 100 mM after about eight days of culture. In some embodiments, the cyanobacterium is Synechocystis and the construct is pPETPDC. In some embodiments, the copper ion responsive promoter is a pPetE promoter. In some embodiments, the construct is integrated into the cyanobacteria chromosome.

### INCORPORATION BY REFERENCE

All publications and patent applications mentioned in this specification are herein incorporated by reference to the same extent as if each individual publication or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 depicts the construction of plasmid pPDC1.

FIG. 2 depicts the construction of the transformation vector pPETPDC.

FIG. 3 depicts the metabolic map for ethanol-producing Synechocystis. pdc gene transformation enables the carbon flux toward ethanol production. Adh gene exists in the cell.

FIG. 4 depicts the experimental observation from an outdoor photobioreactor system for cyanobacterial growth for ethanol production. FIG. 4(a) shows a temperature profile; (b) optical density and ethanol concentration.

### DETAILED DESCRIPTION OF THE INVENTION

### Ethanol Production by Cyanobacteria

Nucleic acid sequences, vectors, host cells, and methods for the production of high levels of ethanol by cyanobacteria are disclosed in accordance with preferred embodiments of the present invention.

Ethanol production from cyanobacteria using sunlight, CO₂, and inorganic nutrients (possibly diverted from a wastewater stream) is an attractive pathway for obtaining a renewable fuel. By combining both the carbon fixation and ethanol generating pathways into a single organism, the costs associated with plant growth/harvesting/processing are circumvented, reducing total input energy, and increasing net energy gain. In contrast to biomass ethanol production processes, the disclosed methods will directly utilize large quantities of CO₂ as a carbon source for fuel production and will thus help reduce this greenhouse gas from the atmosphere.

There are numerous benefits from producing ethanol using photosynthetic microorganisms such as Synechocystis, including: economic opportunities for biofuel production, positive environmental impacts, reduction in global warming, and improved food security. The present methods for producing ethanol from solar energy and CO₂ using cyanobacteria offer significant savings in both capital and operation costs, in comparison to the biomass-based ethanol production facilities. The decreased expenditure is achieved by factors such as: simplified production processes, absence of agricultural crops and residues, no solid wastes to be treated, no enzymes needed, etc. The cyanobacteria fermentation involves no hard cellulose or hemicellulose which is difficult to treat. As a result, there will be no emissions of hazardous air pollutants and volatile organic compounds from cyanobacterial ethanol production plants.

In comparison to traditional methods for biomass ethanol production, the disclosed methods and systems will help preserve agricultural space for food production. Furthermore, cyanobacterial ethanol production plants can be highly distributed without geographical limits because they do not require grain transportation to certain locations or pretreatment of the raw material. The infrastructure and equipment required for ethanol production using the presently disclosed systems are projected to be significantly less than those required for current yeast fermentation technology, allowing for smoother integration with fuel transportation and distribution platforms.

The initial product of photosynthetic fixation of carbon dioxide is 3-phosphoglycerate. 3-phosphoglycerate is used in the Calvin Cycle to regenerate ribulose-1,5-biphosphate, which is the acceptor of carbon dioxide. There are two major branching points where the intermediates of the Calvin Cycle are connected to other metabolic pathways. At one point, fructose-6-phosphate is converted into glucose-6-phosphate and glucose-phosphate, which are the substrates for the pentose phosphate pathway, the synthesis of cellulose (a major component of the cell wall) and the synthesis of glycogen (the major form of carbohydrate reserve). At the other branching point, 3-phosphoglycerate is converted into 2-phosphoglycerate, phosphoenolpyruvate and pyruvate in a sequence of reactions catalysed by phosphoglycerate mutase, enolase and pyruvate kinase, respectively. Pyruvate is directed to the partial TCA cycle for the synthesis of amino acids, nucleotides, etc. in aerobic conditions. Pyruvate is also the substrate for ethanol synthesis.

To convert the carbohydrate reserves into ethanol, the carbohydrate reserves must be diverted to the glycolytic pathway. The presumed pathway for carbohydrate reserve metabolism in cyanobacteria is through both the glycolytic pathway and the phosphogluconate pathway. For the purposes of ethanol formation, the glycolytic pathway is of primary importance. Although not well characterized in cyanobacteria, glycogen is presumed to be metabolized into glucose 1-phosphate by a combination of glycogen phosphorylase and a 1,6-glycosidase. Phosphoglucomutase, phosphoglucoisomerase and phosphofructokinase convert glucose 1-phosphate into a molecule of fructose 1,6-bisphosphate. This compound is cleaved by the action of aldolase and triose phosphate isomerase into two molecules of glyceraldehyde 3-phosphate. This compound is converted into pyruvate through a sequential series of reactions catalysed by glyceraldehyde 3-phosphate dehydrogenase, phosphoglycerate kinase, phosphoglycerate mutase, enolase and pyruvate kinase, respectively.

In some algae and cyanobacteria strains, a small amount of ethanol is synthesized as a fermentation product under dark and anaerobic conditions (Van der Oost et al., “Nucleotide sequence of the gene proposed to encode the small subunit of the soluble hydrogenase of the thermophilic unicellular cyanobacterium Synechococcus PCC 6716.” Nucleic Acids Res. 1989 Dec. 11; 17(23):10098, incorporated herein by reference in its entirety). However, the dark-anaerobic fermentation process is generally operating at a very low level, only sufficient for the survival of the organisms under such stress conditions. The synthesis of ethanol under dark and anaerobic conditions is dependent on the degradation of glycogen reserve, as described above. Moreover, it has been found that ethanol synthesis under anaerobic conditions is totally inhibited by light. Thus, in photosynthetic microorganisms ethanol synthesis is not coupled with photosynthesis and can actually be inhibited by photosynthesis.

Therefore, it has been observed that cyanobacteria do not utilize carbon dioxide to produce ethanol. Furthermore, there are no known photosynthetic microorganisms, including genetically engineered photosynthetic microorganisms, which produce ethanol in relatively substantial amounts. A further complication is that some photosynthetic organisms have been shown to be inhibited by ethanol such that the addition of ethanol to the culture medium inhibits the expression of genes involved in photosynthesis.

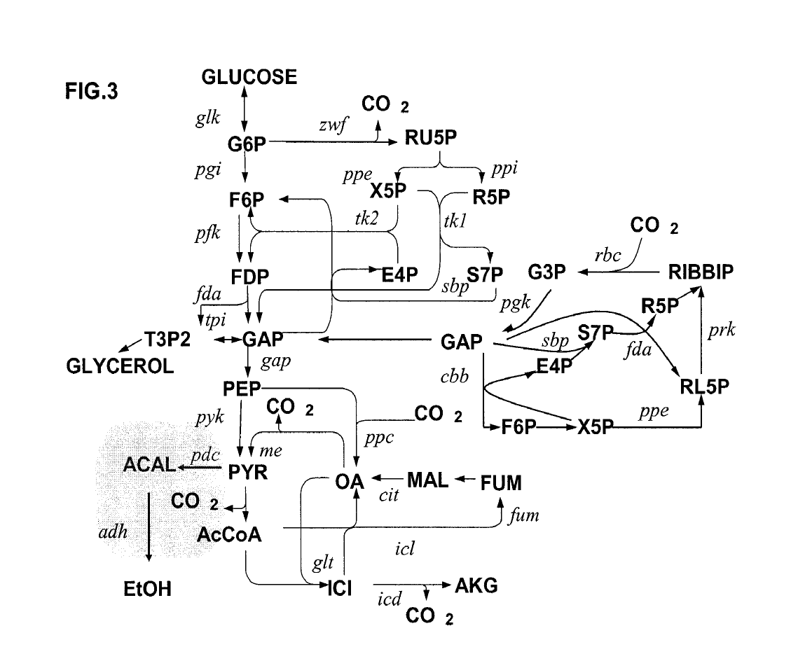

In the present invention, it has been found that cyanobacteria can be successfully genetically engineered to produce a quantifiable amount of ethanol as opposed to utilizing a glycogen reserve as is done under anaerobic and dark conditions. Inorganic carbon is assimilated and is used for both cellular growth and for the production of ethanol via the insertion of the ethanol generating metabolic pathway consisting of the enzyme pdc. The ethanol producing pathway in the high ethanol-tolerant cyanobateria is depicted in FIG. 3.

In some embodiments, the host cell is capable of producing ethanol in recoverable quantities greater than 50 mM ethanol after about 8 days of fermentation. In some embodiments, the amount of ethanol produced after about 8 days of fermentation is about 10 mM, 20 mM, 30 mM, 40 mM, 60 mM, 70 mM, 80 mM, 90 mM, 100 mM or greater.

### Pdc Enzyme

“Pyruvate decarboxylase” and “pdc” refer to an enzyme that catalyzes the decarboxylation of pyruvic acid to acetaldehyde and carbon dioxide. A “pdc gene” refers to the gene encoding an enzyme that catalyzes the decarboxylation of pyruvic acid to acetaldehyde and carbon dioxide.

In anaerobic conditions, pdc is part of the fermentation process that occurs in yeast, especially of the Saccharomyces genus, to produce ethanol alcohol by fermentation. Pyruvate decarboxylase starts this process by converting pyruvate into acetaldehyde and carbon dioxide (Tadhg P. Begley; McMurry, John 2005 pp. page 179). To do this, two thiamine pyrophosphate (TPP) and two magnesium ions are required as a cofactor.

### Genetically Engineered Cyanobacteria

In one aspect, the present invention provides a genetically engineered cyanobacterium comprising a construct comprising polynucleotide sequences encoding pyruvate decarboxylase (pdc), wherein the cyanobacterium is capable of producing ethanol in a quantity that is greater than about 50 mM ethanol after about 8 days of fermentation. The cyanobacteria used in this invention possess tolerance to high temperature and high ethanol concentrations.

In some embodiments, the cyanobacterium is the strain Synechocystis. In some embodiments, the cyanobacterium is Synechocystis sp. PCC 6803, or other transformable strain of Synechocystis. In some embodiments, the cyanobacterium is a wild-type strain of Synechocystis sp. PCC 6803. In some embodiments, the cyanobacterium is Synechococcus PCC 7942, or other transformable strain of Synechococcus.

Through directed evolution of the cyanobacterium Synechocystis sp PCC 6803 (purchased from the United States American Type Culture Collection (ATCC), ATCC® Number: 27184 ™), a Synechocystis mutant which possesses high temperature, high ethanol resistance. The mutant is named Synechocystis strictus (referred to as S. strictus).

For all the microorganisms capable of ethanol production (such as E. coli, yeast Sacchromyces cerevisaie, Zymomonas mobilis), there is a common pathway from the end product of glycolytic pathway, pyruvate, through the intermediate metabolite, acetaldehyde to ethanol. The reaction step of pyruvate to acetaldehyde is catalyzed by the enzyme pyruvate decarboxylase. The reaction step from acetaldehyde to ethanol is catalyzed by the enzyme alcohol dehydrogenase. Cyanobacteria are among the oldest forms of life on the earth, appearing in the fossil record as much as 3.5 billion years ago. This group of photosynthetic microorganisms is able to survive harsh environmental conditions, such as high-temperature, ice, lack of oxygen, dry and high salinity, strong radiation and other harsh living conditions. There is no pyruvate decarboxylase (pdc) gene in the Synechocystis PCC 6803 genome, so its metabolic pathway for ethanol production is incomplete. As a result, wild type Synechocystis PCC 6803 does not have ethanol production capacity.

In one aspect, the present invention discloses a genetic engineering method to transform a blue-green algae (referred to as cyanobacteria), so that the cyanobacteria are able to convert carbon dioxide directly into ethanol upon copper ion e.g. copper sulfate induction. The cyanobacterium Synechocystis sp. PCC 6803 (hereinafter referred to as “sp-6803”) used in this invention is a non-filament and non-nitrogen-fixing, fresh water strain, capable of both autotrophic and heterotrophic growth. Synechocystis PCC 6803 is the first photosynthetic organism for which the genome was completely sequenced (Ikeuchi et al., Tanpakushitsu Kakusan Koso 1996, 41 (16): 2579-2583). This has laid an important foundation for the development of genetic engineering on microalage/cyanobacteria. Synechocystis 6803 is able to integrate foreign DNA into its chromosome by the utilization of homologous recombination technology. The genomic information, coupled with the rich biochemistry and physiological information available for Synechocystis sp. PCC 6803, has made this strain one of the most popular organisms for genetic and physiological studies of photosynthesis for higher plant systems. (Aoki et al., J Microbiol Methods 2002, 49 (3): 265-74., Vermaas et al., Proc Natl Acad Sci USA, 1986, 83 (24): 9474-9477, Williams, Methods Enzymol., 1988, 167:766-778).

In one aspect, the present invention provides a nucleic acid construct comprising a copper ion inductive promoter, and a sequence encoding a pyruvate decarboxylase (pdc) enzyme. In some embodiments, the present invention discloses the construction of recombinant plasmid from Acetobacter pasteurianus, the construction of the gene expression vector pPETPDC, and directed evolution by “heat shock” and screening of resultant Synechocystis mutant S. strictus, insertion of the pdc gene into S. strictus so as to create the novel ethanol production pathway. S. strictus is thus highly resistant to temperature elevation and ethanol accumulation in the growth media. Ethanol production can be induced by the addition of copper ion into the growth media. It thus integrates photosynthesis, carbon dioxide collection and the production of biofuels within one production host.

The present invention uses the “directed evolution” approach to improve the heat and ethanol tolerance of Synechocystis 6803. The consumption of carbon dioxide (CO₂) as a carbon source for large-scale production of fuel ethanol requires cyanobacterial cell growth in the outdoor closed-system photobioreactors to meet the needs for cellular photosynthesis. Suitable temperature for cell growth is around 30° C. In reality, the outdoor temperature in the summer time may be above 40° C. in certain areas of the US. This would have negative impacts on the normal growth of algae cells and ethanol production. For example, wild type Syencocystis 6803 cells is grown using BG-11 media in an outdoor photobioreactor with no temperature control. The cell growth became “bleached”, since the elevated temperature damaged the chlorophyll and disactivate the photosynthetic apparatus of cyanobacteria. In another example, the wild type Synechocystis 6803 is grown in the laboratory photobioreactor with temperature controlled at 30° C. 50 ml is sampled from the photobioreactor and put into a 150 ml tube. The tube is then shaken in a 45° C. water bath for “heat shock” for two hours. It is then transferred back into the 30° C. shaker for batch culture. Three days later, the color of the cell culture is changed from green to white, indicating that the “bleaching” occurred in the cells.

In some embodiments, the present invention uses a “directed evolution” means to enhance the ability of cyanobacteria to tolerate temperature elevation and ethanol accumulation in the media, it thus avoids the need for temperature control. When the cells have increased their tolerance of heat, they are also more resistant to higher ethanol concentration in the media.

“Directed evolution” can be implemented for Synechocystis PCC 6803 to derive not only temperature resistance, but also ethanol tolerance. One of the major issues for cyanobacteria to be used for ethanol production is that when ethanol in the culture medium accumulates to a certain degree of concentration (for example, 5% v/v concentration), it will hinder the growth of cyanobacteria. Cyanobacteria might start to die at higher concentrations of ethanol. It is of critical importance for cyanobacteira to increase its tolerance to elevated ethanol concentration. The latest research results show that the changes in growth conditions, such as temperature elevation, ethanol accumulation in the media, and other adverse external factors, will cause the cells to be stressed. Consequently, the cells will respond with over-expression of so-called “heat shock” proteins (Roy et al., JOURNAL OF BACTERIOLOGY, 180 (15): 3997-4001, 1998; Glatz et al, Acta Biologica Szegediensis. 46 (3-4): 53, 2002). Furthermore, the scientific findings also show that there exists an interaction between heat and ethanol tolerance (Michel et al., JOURNAL OF BACTERIOLOGY, 165 (3):1040-1042, 1986). When the heat resistance for E. coli, yeast and cyanobacteira cells increases, their ethanol tolerance will increase as well (Horvath et al., Biochemistry, 95:3513-3518, 1998). Our experimental observations indicate that wild type Synechocystis may become “bleached” three days after addition of ethanol for the media to have 5% ethanol concentration. In comparison, the same ethanol feeding did not hinder the S. Strictus growth. The Synechocystis mutant created by the directed evolution approach described in the present invention is named Synechocystis strictus (referred to as S. strictus).

### Expression Vector and Cyanobacteria Transformation

Nucleic acids and recombinant expression vectors for the optimization of ethanol production are disclosed in accordance with some embodiments of the present invention. A “promoter” is an array of nucleic acid control sequences that direct transcription of an associated polynucleotide, which may be a heterologous or native polynucleotide. A promoter includes nucleic acid sequences near the start site of transcription, such as a polymerase binding site. The promoter also optionally includes distal enhancer or repressor elements which can be located as much as several thousand base pairs from the start site of transcription.

“Polynucleotide” and “nucleic acid”, which are used interchangeably herein, refer to a polymer composed of nucleotide units (ribonucleotides, deoxyribonucleotides, related naturally occurring structural variants, and synthetic non-naturally occurring analogs thereof) linked via phosphodiester bonds, related naturally occurring structural variants, and synthetic non-naturally occurring analogs thereof. Thus, the term includes nucleotide polymers in which the nucleotides and the linkages between them include non-naturally occurring synthetic analogs. It will be understood that, where required by context, when a nucleotide sequence is represented by a DNA sequence (i.e., A, T, G₅ C), this also includes an RNA sequence (i.e., A, U, G, C) in which “U” replaces “T.”

“Recombinant” refers to polynucleotides synthesized or otherwise manipulated in vitro (“recombinant polynucleotides”) and to methods of using recombinant polynucleotides to produce gene products encoded by those polynucleotides in cells or other biological systems. For example, a cloned polynucleotide may be inserted into a suitable expression vector, such as a bacterial plasmid, and the plasmid can be used to transform a suitable host cell. A host cell that comprises the recombinant polynucleotide is referred to as a “recombinant host cell” or a “recombinant bacterium.” The gene is then expressed in the recombinant host cell to produce, e.g., a “recombinant protein.” A recombinant polynucleotide may serve a non-coding function (e.g., promoter, origin of replication, ribosome-binding site, etc.) as well.

The term “homologous recombination” refers to the process of recombination between two nucleic acid molecules based on nucleic acid sequence similarity. The term embraces both reciprocal and nonreciprocal recombination (also referred to as gene conversion). In addition, the recombination can be the result of equivalent or non-equivalent cross-over events. Equivalent crossing over occurs between two equivalent sequences or chromosome regions, whereas nonequivalent crossing over occurs between identical (or substantially identical) segments of nonequivalent sequences or chromosome regions. Unequal crossing over typically results in gene duplications and deletions. For a description of the enzymes and mechanisms involved in homologous recombination see, Watson et al.,—Molecular Biology of the Gene pp 313-327, The Benjamin/Cummings Publishing Co. 4th ed. (1987).

The term “non-homologous or random integration” refers to any process by which DNA is integrated into the genome that does not involve homologous recombination. It appears to be a random process in which incorporation can occur at any of a large number of genomic locations.

A “heterologous polynucleotide sequence” or a “heterologous nucleic acid” is a relative term referring to a polynucleotide that is functionally related to another polynucleotide, such as a promoter sequence, in a manner so that the two polynucleotide sequences are not arranged in the same relationship to each other as in nature. Heterologous polynucleotide sequences include, e.g., a promoter operably linked to a heterologous nucleic acid, and a polynucleotide including its native promoter that is inserted into a heterologous vector for transformation into a recombinant host cell. Heterologous polynucleotide sequences are considered “exogenous” because they are introduced to the host cell via transformation techniques. However, the heterologous polynucleotide can originate from a foreign source or from the same source. Modification of the heterologous polynucleotide sequence may occur, e.g., by treating the polynucleotide with a restriction enzyme to generate a polynucleotide sequence that can be operably linked to a regulatory element. Modification can also occur by techniques such as site-directed mutagenesis.

An “expression cassette” or “construct” refers to a series of polynucleotide elements that permit transcription of a gene in a host cell. Typically, the expression cassette includes a promoter and a heterologous or native polynucleotide sequence that is transcribed. Expression cassettes or constructs may also include, e.g., transcription termination signals, polyadenylation signals, and enhancer elements.

The term “operably linked” refers to a functional relationship between two parts in which the activity of one part (e.g., the ability to regulate transcription) results in an action on the other part (e.g., transcription of the sequence). Thus, a polynucleotide is “operably linked to a promoter” when there is a functional linkage between a polynucleotide expression control sequence (such as a promoter or other transcription regulation sequences) and a second polynucleotide sequence (e.g., a native or a heterologous polynucleotide), where the expression control sequence directs transcription of the polynucleotide.

“Competent to express” refers to a host cell that provides a sufficient cellular environment for expression of endogenous and/or exogenous polynucleotides.

All numbers expressing quantities of ingredients, reaction conditions, and so forth used in the specification and claims are to be understood as being modified in all instances by the term “about.” Accordingly, unless indicated to the contrary, the numerical parameters set forth in the specification and attached claims are approximations that can vary depending upon the desired properties sought to be obtained by the present invention. At the very least, and not as an attempt to limit the application of the doctrine of equivalents to the scope of the claims, each numerical parameter should be construed in light of the number of significant digits and ordinary rounding approaches.

Nucleic acids and recombinant expression vectors for the optimization of ethanol production are disclosed in accordance with some embodiments of the present invention. Example 1 shows one embodiment of a system that can be used to perform a variety of methods or procedures. The present invention uses the molecular cloning technology to integrate pyruvate decarboxylase (pdc) gene sequence (SEQ. ID NO: 3), and copper ion inducible pPetE promoter (SEQ. ID NO: 1) into the expression vector pPetE, to create the recombinant plasmid, and then to transform the genes into S. strictus to construct a recombinant mutant which can be induced by copper ion, for example, copper sulfate, to produce ethanol via efficient use of carbon dioxide. The pPetE vector is used to integrate these genes under the control of the pPetE copper responsive promoter in the cyanobacterial genome.

A recombinant expression vector for transformation of a host cell and subsequent integration of the gene(s) of interest is prepared by first isolating the constituent polynucleotide sequences, as discussed herein. In some embodiments, the gene(s) of interest are homologously integrated into the host cell genome. In other embodiments, the genes are non-homologously integrated into the host cell genome. Preferably, the gene(s) of interest are homologously integrated into the Synechocystis genome. In some embodiments, the pPetE vector integrates into the Synechocystis genome via double homologous recombination. The polynucleotide sequences, e.g., a sequence encoding the pdc enzymes driven by a promoter, are then ligated to create a recombinant expression vector, also referred to as a “pdc construct,” suitable for transformation of a host cell. Methods for isolating and preparing recombinant polynucleotides are well known to those skilled in the art. Sambrook et al., Molecular Cloning. A Laboratory Manual (2d ed. 1989); Ausubel et al., Current Protocols in Molecular Biology (1995)), provide information sufficient to direct persons of skill through many cloning exercises.

One preferred method for obtaining specific polynucleotides combines the use of synthetic oligonucleotide primers with polymerase extension or ligation on a mRNA or DNA template. Such a method, e.g., RT, PCR, or LCR, amplifies the desired nucleotide sequence (see U.S. Pat. Nos. 4,683,195 and 4,683,202). Restriction endonuclease sites can be incorporated into the primers. Amplified polynucleotides are purified and ligated to form an expression cassette. Alterations in the natural gene sequence can be introduced by techniques such as in vitro mutagenesis and PCR using primers that have been designed to incorporate appropriate mutations. Another preferred method of isolating polynucleotide sequences uses known restriction endonuclease sites to isolate nucleic acid fragments from plasmids. The genes of interest can also be isolated by one of skill in the art using primers based on the known gene sequence.

Promoters suitable for the present invention include any suitable copper ion-responsive promoter such as, for example, the pPetE promoter. The promoter of the petE gene, encoding the protein plastocyanin, has been shown to respond to copper added to the medium in which the cyanobacterium Anabaena PCC 7120 is growing (Ghassemian, M; et al. Microbiology. 1994; 140:1151-1159). In some embodiments, the construct vector further comprises a polynucleotide comprising a copper ion responsive gene. The expression from the petE promoter is smoothly induced depending on the amount of copper supplied.

In some embodiments, the promoter comprises the Synechococcus pPetE promoter sequence shown in SEQ ID NO: 1. In SEQ ID NO: 1, the TCC at the 3′ terminus of the wild type pPetE promoter is replaced with the sequence CAT in order to generate an NdeI restriction site at the start codon while maintaining the spatial integrity of the promoter/ORF construct. This allows for the creation of a system whereby the gene(s) of interest may be expressed via induction by addition of copper ion to the culture media. Copper sulfate may be used as the copper source for growth prior to induction (W J. Buikema and R. Haselkorn, Proc Natl Acad Sci USA. 2001 Feb. 27; 98(5): 2729-2734).

Any pdc gene capable of being expressed may be used in the present invention. In some embodiments, the pdc gene is the Zymomonas mobilis pdc gene. In some embodiments, the pdc gene is obtained from the Zymomonas mobilis plasmid pLOI295. In some embodiments, the pdc gene comprises the nucleic acid sequence shown in SEQ ID NO: 3 from Acetobacter pasteurianus. In some embodiments, the pdc gene is a nucleic acid sequence encoding the protein shown in SEQ ID NO: 8. In other embodiments, the pdc gene is a nucleic acid encoding the pdc enzyme obtained from Zymobacter paimae. There are other sources of pdc enzyme including Saccharomyces cerevisciae.

The isolated polynucleotide sequence of choice, e.g., the pdc gene driven by the promoter sequence discussed above, is inserted into an “expression vector,” “cloning vector,” or “vector,” terms which usually refer to plasmids or other nucleic acid molecules that are able to replicate in a chosen host cell. Expression vectors can replicate autonomously, or they can replicate by being inserted into the genome of the host cell.

Often, it is desirable for a vector to be usable in more than one host cell, e.g., in E. coli for cloning and construction, and in, e.g., Synechocystis for expression. Additional elements of the vector can include, for example, selectable markers, e.g., kanamycin resistance or ampicillin resistance, which permit detection and/or selection of those cells transformed with the desired polynucleotide sequences.

The particular vector used to transport the genetic information into the cell is also not particularly critical. Any suitable vector used for expression of recombinant proteins can be used. In preferred embodiments, a vector that is capable of being inserted into the genome of the host cell is used. In some embodiments, the vector is pPetE. Expression vectors typically have an expression cassette that contains all the elements required for the expression of the polynucleotide of choice in a host cell. A typical expression cassette contains a promoter operably linked to the polynucleotide sequence of choice. The promoter used to direct expression of pdc is as described above, and is operably linked to a sequence encoding the pdc protein. The promoter is preferably positioned about the same distance from the heterologous transcription start site as it is from the transcription start site in its natural setting. As is known in the art, however, some variation in this distance can be accommodated without loss of promoter function.

After construction and isolation of the recombinant expression vector, it is used to transform a host cell for ethanol production. The particular procedure used to introduce the genetic material into the host cell for expression of a protein is not particularly critical. Any of the well known procedures for introducing foreign polynucleotide sequences into host cells can be used. In some embodiments, the host cells can be transformed and screened sequentially via the protocol described by Williams (1988). This method exploits the natural transformability of the Synechocystis sp. PCC 6803 cyanobacteria, where transformation is possible via simple incubation of purified plasmid construct with exponentially growing cells. In some embodiments, the cyanobacterium is Synechocystis sp. PCC 6803 or other transformable strain of Synechocystis. In some embodiments, the cyanobacterium is a wildtype strain of Synechocystis sp. PCC 6803. In some embodiments, the cyanobacterium is Synechococcus PCC 7942 or other transformable strain of Synechococcus.

Host cells for transformation with the recombinant expression vector described above include any suitable host cyanobacterium competent to produce ethanol, especially members of the genus Synechocystis. Host cells suitable for use in the present invention include, for example, wild type Synechocystis sp. PCC 6803 and a mutant Synechocystis created by Howitt et al. (1999) that lacks a functional NDH type 2 dehydrogenase (NDH-2(−)). The type 2 dehydrogenase is specific for the regeneration of NAD+ from NADH. Flux through the ethanol pathway may be increased in the mutant. In particularly preferred embodiments, the host cells are Synechocystis. Host cells that are transformed with the pdc construct are useful recombinant cyanobacteria for production of ethanol. Preferred subspecies of Synechocystis include, e.g., Synechocystis PCC 6803. A preferred strain is the Synechocystis sp. PCC 6803 NDH-2(−) mutant.

After the host cell is transformed with the pdc construct, the host cell is incubated under conditions suitable for production of ethanol. Typically, the host cell will be grown in a photoautotrophic liquid culture in BG-I 1 media, with a 1 L/min air sparge rate and a pH setpoint of 8.5, controlled via sparging with CO₂, and the temperature maintained at 30° C. Various media for growing cyanobacteria are known in the art. In some embodiments, Synechocystis sp. PCC 6803 is cultured on standard BG-1 1 media plates, with or without the addition of (final concentration): 5 mM glucose, 5% sucrose, and/or either 5 μml″¹, 25 μml″¹, or 50 μg ml″¹ kanamycin. Plates containing Synechocystis sp. PCC 6803 are incubated at 30° C. under ˜100 microeinsteins m² s^{˜1}. All Synechocystis liquid cultures are grown in standard BG-1 1, with the addition of 50 μg ml″¹ kanamycin when appropriate.

In this invention, a copper inducible pPetE promoter is used to achieve a stable and efficient gene expression for the improvement of ethanol production efficiency. The system chosen is based on the observation of Straus and coworkers that transcription of the gene encoding the copper protein plastocyanin in the cyanobacterium Synechococcus PCC 7942 is regulated by copper (Ghassemian, M; et al. Microbiology. 1994; 140:1151-1159). The petE promoter may be amplified by PCR using the following two primers: 5′-GGATC CCAGT ACTCA GAATT TTTTG CT-3′ and 5′-GAATT CCATG GCGTT CTCCT AACCT G-3′. The resulting 372-bp fragment is blunt-end cloned into the HincII site of pUC19 to generate pPetE promoter sequence (William J. Buikema and Robert Haselkom, Proc Natl Acad Sci USA. 2001 Feb. 27; 98(5): 2729-2734).

The ethanol gene expression is not affected by changes in temperature and lighting intensity. In addition, the “heat shock” directed evolution has been introduced to obtain higher heat and ethanol tolerance. The present invention not only discloses the use of photobioreactors in the lab, but also the use of outdoor photobioreactors for fermentation. The results from the outdoor experimental device have yielded a much higher amount of ethanol. In some embodiments, the host cell is capable of producing ethanol in recoverable quantities greater than 50 mM ethanol after about 8 days of fermentation. In some embodiments, the amount of ethanol produced after about 8 days of fermentation is about 10 mM, 20 mM, 30 mM, 40 mM, 60 mM, 70 mM, 80 mM, 90 mM, 100 mM or greater.

In order to obtain the ethanol production capacity, in some embodiments, the cyanobacteria are transformed to encode pyruvate decarboxylase (pdc) enzyme. It should be understood that in one embodiment, the invention uses a specific pdc gene and a copper inducible pPetE promoter sequence for the ethanol production by Synechocystis. The invention encompasses the use of other sequences encoding pdc gene with the same function and the polynucleotide sequences are not limited to SEQ. ID NO: 3 disclosed herein as an example.

For example, the invention described in the pyruvate decarboxylase gene, as well as copper ion induced pPetE promoter sequence also contains the following multi-nucleotide, and with its SEQ. ID NO: 3 or SEQ. ID NO: 1 base sequence complementary base sequence group into a multi-nucleotide hybrid, under strict conditions and with pyruvate decarboxylase activity or copper ion-induced promoter activity of the multi-nucleotide, or its serial number, and contains: 1, or serial number: 3 sequence composition Multi-nucleotide hybrid strict conditions and with pyruvate decarboxylase activity or copper ion-induced promoter activity of the chemical nucleotide.

Here “in the strict conditions of the multi-nucleotide hybrid” refers to the SEQ. ID NO: 3 or SEQ. ID NO: 1 of the base sequence complementary base sequence of nucleotides comprising more than for all or part of the probe, Using colony hybridization, or plaque hybridization Southern hybridization, and so get more nucleotides (such as DNA). Hybrid methods, such as the use of Molecular Cloning 3rd Ed., Current Protocols in Molecular Biology, John Wiley & Sons 1987-1997, and so described.

For this statement as described as “strict conditions”, such as 5×SSC, 5×Denhardt solution, 0.5% SDS, 50% formamide, 32° C. conditions; or, for example, 5×SSC, 5×Denhardt solution, 0.5% SDS, 50% formamide, 42° C. conditions; or, for example, for 5×SSC, 5×Denhardt solution, 0.5% SDS, 50% formamide, 50° C. Under these conditions, the more the temperature is raised, the more efficient it is to have access to the high number of nucleotide homology (such as DNA). Hybrid impact of stringent factor for the temperature, concentration of probe, probe length, ionic strength, time, concentration and other factors, the technical staff in the field through the appropriate choice of these factors can achieve the same strict conditions. It needs to be noted that in addition to fuel ethanol, other uses of ethanol are contemplated within the scope of the present invention.

Enhanced secretion of ethanol is observed after host cells competent to produce ethanol are transformed with the pdc construct and the cells are grown under suitable conditions as described above, for example, in media containing copper ion for ethanol induction. Enhanced secretion of ethanol may be observed by standard methods, discussed more fully below in the Examples, known to those skilled in the art. In some embodiments, the host cells are grown using batch cultures. In some embodiments, the host cells are grown using photobioreacter fermentation. In some embodiments, the host cells are grown in a Celligen® Reactor. In some embodiments, the growth medium in which the host cells are grown is changed, thereby allowing increased levels of ethanol production. The number of medium changes may vary. Ethanol concentration levels may reach from about 20 mM to about 100 mM after about 8 days of fermentation. In some embodiments, ethanol concentration levels may reach from about 20 to about 100 mM after 8 days of fermentation. In some embodiments, the ethanol production level is about 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 mM or greater than 100 mM after about 8 days of fermentation. In cases where the medium is changed, in some embodiments, the ethanol production level is about 25.0, 25.1, 25.2, 25.3, 25.4, 25.5, 25.6, 25.7, 25.8, 25.9, 26.0, 26.1, 26.2, 26.3, 26.5, 26.6, 26.7, 26.8, 26.9, 27.0, 27.1, 27.2, 27.3, 27.5, 27.6, 27.7, 27.8, 27.9, 28.2, 28.2, 28.3, 28.5, 28.6, 28.7, 28.8, 28.9, 29.0, 29.1, 29.2, 29.3, 29.5, 29.6, 29.7, 29.8, 29.9, 30.0, 30.1, 30.2, 30.3, 30.4, 30.5, 30.6, 30.7, 30.8, 30.9, 31.0, 31.1, 31.2, 31.3, 31.4, 31.5, 31.6, 31.7, 31.8, 31.9, 32.0, 32.1, 32.2, 32.3, 32.4, 32.5, 32.6, 32.7, 32.8, 32.9, 33.0, 33.1, 33.2, 33.3, 33.4, 33.5, 33.6, 33.7, 33.8, 33.9, 34.0, 34.1, 34.2, 34.3, 34.4, 34.5, 34.6, 34.7, 34.8, 34.9, 35.0, 35.1, 35.2, 35.3, 35.4, 35.5, 35.6, 35.7, 35.8, 35.9, 36.0, 36.1, 36.2, 36.3, 36.5, 36.6, 36.7, 36.8, 36.9, 37.0, 37.1, 37.2, 37.3, 37.5, 37.6, 37.7, 37.8, 37.9, 38.2, 38.2, 38.3, 38.5, 38.6, 38.7, 38.8, 38.9, 39.0, 39.1, 39.2, 39.3, 39.5, 39.6, 39.7, 39.8, 39.9, 40.0, 40.1, 40.2, 40.3, 40.4, 40.5, 40.6, 40.7, 40.8, 40.9, 41.0, 41.1, 41.2, 41.3, 41.4, 41.5, 41.6, 41.7, 41.8, 41.9, 42.0, 42.1, 42.2, 42.3, 42.4, 42.5, 42.6, 42.7, 42.8, 42.9, 43.0, 43.1, 43.2, 43.3, 43.4, 43.5, 43.6, 43.7, 43.8, 43.9, 44.0, 44.1, 44.2, 44.3, 44.4, 44.5, 44.6, 44.7, 44.8, 44.9, 45.0, 45.1, 45.2, 45.3, 45.4, 45.5, 45.6, 45.7, 45.8, 45.9, 46.0, 46.1, 46.2, 46.3, 46.5, 46.6, 46.7, 46.8, 46.9, 47.0, 47.1, 47.2, 47.3, 47.5, 47.6, 47.7, 47.8, 47.9, 48.2, 48.2, 48.3, 48.5, 48.6, 48.7, 48.8, 48.9, 49.0, 49.1, 49.2, 49.3, 49.5, 49.6, 49.7, 49.8, 49.9 or 50.0 mM after about 5 days of fermentation. The fermentation times may vary from about 2 days to about 30 days of fermentation. In some embodiments, the fermentation time is about 2, 3, 4, 5, 6, 7, 8, 9, 10, 11, 12, 13, 14, 15, 16, 17, 18, 19, 20, 21_{S} 22, 23, 24 or 25 days.

The air sparge rate during host cell growth may be from 0.1 L/min to 3.0 L/min. In some embodiments, the air sparge rate during host cell growth is about 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9 or 3.0 L/min. Preferably, the air sparge rate is 1 L/min. The pH setpoint for host cell growth may be from 7.0 to 9.5. In some embodiments, the pH setpoint is about 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, or 9.5. The temperature during host cell growth may be from about 25° C. to 35° C. In some embodiments, the temperature is about 25.0, 25.1, 25.2, 25.3, 25.4, 25.5, 25.6, 25.7, 25.8, 25.9, 26.0, 26.1, 26.2, 26.3, 26.5, 26.6, 26.7, 26.8, 26.9, 27.0, 27.1, 27.2, 27.3, 27.5, 27.6, 27.7, 27.8, 27.9, 28.2, 28.2, 28.3, 28.5, 28.6, 28.7, 28.8, 28.9, 29.0, 29.1, 29.2, 29.3, 29.5, 29.6, 29.7, 29.8, 29.9, 30.0, 30.1, 30.2, 30.3, 30.4, 30.5, 30.6, 30.7, 30.8, 30.9, 31.0, 31.1, 31.2, 31.3, 31.4, 31.5, 31.6, 31.7, 31.8, 31.9, 32.0, 32.1, 32.2, 32.3, 32.4, 32.5, 32.6, 32.7, 32.8, 32.9, 33.0, 33.1, 33.2, 33.3, 33.4, 33.5, 33.6, 33.7, 33.8, 33.9, 34.0, 34.1, 34.2, 34.3, 34.4, 34.5, 34.6, 34.7, 34.8, 34.9 or 35.0° C.

While exemplary embodiments of the invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention.

### Example 1

### Generation of High Temperature and High Ethanol Tolerant Cyanobacteria

Photosynthetic bioreactor cluster (modified by a built-in 6 150 ML shaker flask, for the working volume of 50 ML) is inoculated by the wild type Synechocystis PCC 6803. The cells are grown with the BG-11 culture medium. The shaker is equipped with built-in light source, the reactor surface light intensity is about 200 μEinstein/m2/s. Temperature of the shaker is controlled to be at less than 30° C., and the agitation is set at 200 RPM. The culture is put into a hot water bath 5 days after initial cell growth for the heat shock. It is then put back into the photosynthetic bioreactor cluster. About 3 days later, all the cells would become “bleached”. Centrifuge would then be used to separate the cell pellets from the supernatant. Remove the supernatant. Wash the cell pellets and grow the cells with fresh BG-11 culture medium. For those Synechocystis cells which have survived the heat shock, they would regenerate chlorophyll and the color of the culture will become green, this is an indication that they have resumed the ability for cell growth and photosynthesis. During the above-mentioned repeated heat-shock process, the surviving cells would slowly reprogram their metabolism to eventually adapt to the temperature elevation during cell cultures. The above-mentioned repeated process comprising the steps of heat shock, whitening, and recovery for “directed evolution” has been carried out for a year. The procedure comes with a gradual increase in temperature (from 35° C. to 47° C.) and time of heat shock (from 30 Minutes increased to 4 hours). Finally, this approach has resulted in a Synechocystis strain which is able to grow in high temperature conditions (>45° C.).

### Example 2

### Expression Vector Containing Pyruvate Decarboxylase (pdc) Gene and pPetE Promoter

Plasmid/PCR product cleanup kits and Taq DNA polymerase are purchased from Qiagen®. Restriction enzymes, Vent_{R}® Polymerase and T4 DNA ligase are obtained from New England Biolabs®. The plasmid PSBAIIKS is obtained from Dr. Vermaas at Arizona State University. The plasmid LOI295, containing the Z. mobilis pdc gene, is obtained from Dr. Lonnie Ingram at the University of Florida. The petE promoter sequence is artificially synthesized by Realgene® (SEQ.ID.NOs 1 and 2).

Construction of Transformation Plasmid pPDC1

PCR reaction is used for both the amplification of the pdc gene (SEQ. ID. No 3) from pLOI295 and for the simultaneous introduction of NdeI and BamHI sites at the 5′ and 3′ ends, respectively. The following primers are used for the above PCR reaction (restriction sites are underlined, induced mutations are in bold): Upstream: 5′-ggAgTAAgCATATgAgTTATACTg-3′ Downstream: 5′-ggATCTCgACTCTAgAggATCC-3′, with a resultant amplicon of 3117 bp. PCR reaction is carried out as follows: Total reaction volume of 50 μl, 0.36 μg of pLOI295 as template, 4 Units of Vent_{R}® polymerase, a final concentration of 0.5 μM for each primer, 300 μM of each dNTP. The following program is run on an EppendorfMastercycler®: Initial denaturation at 94° C. for 2 min, followed by 35 cycles of 10 s denaturation at 94° C., 1 min annealing at 47° C., and 3.7 min extension at 68° C.; finally, hold at 4° C.

Based on the location and sequence for aphX and sacB genes, the following primers are used for the PCR reaction (restriction sites are underlined, induced mutations are in bold, PCR primer P1 (SEQ ID NO: 4) and PCR primer P2 (SEQ ID NO: 5)):

```
P1 (5-end primer):
5′-GGAGTAAGCATATGAGTTATACTG -3′
                          NdeI
P2 (3′end primer):
5′- GGATCTCGACTCTAGAGGATCC -3′
                                                  BamHI
```

Primer P1 is designed to have an NdeI restriction site, as shown as CA ↓TATG, while Primer P2 is designed to have a BamHI restriction site, as shown as GGATC ↓C. A pdc DNA fragment P (1410 bp) is obtained by conventional PCR amplification. PCR reaction is carried out as follows: in an aseptic 0.5 mL centrifuge tubes, add deionized water 36 μl; 10×Taq buffer-5 μl; 4XdNTP (2.5 mmol/L) 5 μl; P1 primer 1 μl; P2 primer 1 μl; template (that is, synthetic pdc) 1 μl; Taq enzyme (3 U/1 μl for a total of 50 μl. The PCR assay utilized the following cycling program: Initial denaturation at 94° C. for 5 minutes.; followed by 30 second denaturation at 94° C.; 45 second annealing at 50° C.; and 50 second extension at 72° C.; followed by 35 cycle; a final 5 minute extension at 72° C.; hold at 4° C.

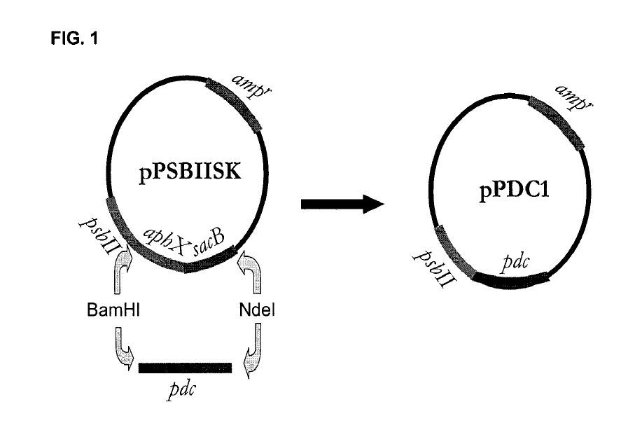

It resulted in the removal of aphX/sacB selection cassette and subcloning of pdc into the backbone of pPSBAIIKS via NdeI/BamHI dual digestion, and in the creation of transformation vector, pMota (FIG. 1). The resultant plasmid is termed pPDC1 (SEQ ID No 6).

Construction of Transformation Vector pPETPDC

The copper induced promoter pPetE is used to replace the light-driven psbAII promoter, so the strain could be induced by the addition of copper ions. The pPetE promoter primer is designed as follows:

```
T1 (5′ primer):
5′-GAGAGAGAGCTGCAGAGCGTTCCAGTGGATATT-3′
                                                        PstI
T2 (3′ primer):
5′-ATTATATATATCATATGTTCATCTGCCTACAAAGCAGC-3′
                                NdeI
```

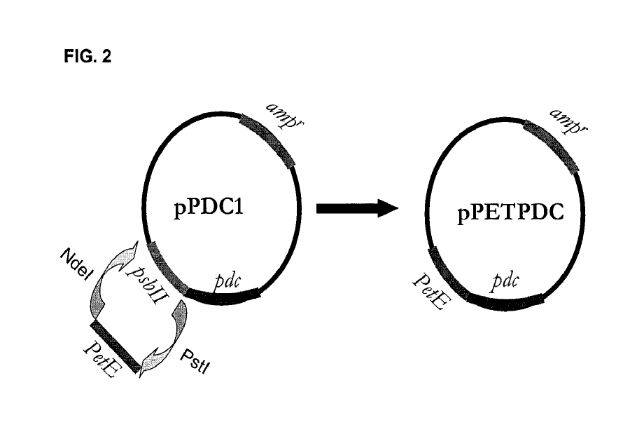

Primer T1 is designed to have a PstI restriction site, as shown as CTGCA ↓G, primers T2 is designed to have an NdeI restriction sites, as shown as CA ↓TATG. Conventional PCR amplification is conducted to obtain a 474 bp fragment for pPetE (FIG. 2).

PCR reaction is carried out as follows: in an aseptic 0.5 mL centrifuge tubes, add deionized water 36 μl; 10×Taq buffer-5 μl; 4XdNTP (2.5 mmol/L) 5 μl; P1 primer 1 μl; P2 primer 1 μl; template (that is, synthetic pdc) 1 μl; Taq enzyme (3 U/μl) 1 μl for a total of 50 μl. The PCR assay utilized the following cycling program: Initial denaturation at 94° C. for 5 minutes.; followed by 30 second denaturation at 94° C.; 45 second annealing at 50° C.; and 50 second extention at 72° C.; followed by 35 cycle; a final 5 minute extension at 72° C.; hold at 4° C.

### Example 3

### Transformation of S. strictus with pdc Gene

After the PCR reaction, 2.5 μl PCR amplification products is taken to run 1% agarose gel electrophoresis (2×TAE, 100V voltage, 40 minutes). There appears a bright band at 1410 bp on the gel electrophoresis image, which confirms that the pdc fragment has been successfully amplified. PCR is used for the simultaneous introduction of NdeI and BamHI sites at the 5′ and 3′ ends, respectively. These sites then allow for subcloning pdc into the backbone of the pPSBAIIKS plasmid, resulting from removal of the aphX/sacB selection cassette via NdeI/BamHI dual digestion, yielding pSKBPDC. T4 DNA ligase is used for the ligation; the plasmid is then transferred into E. coli C600. Through Amp screening, the recombinant plasmid pPDC1 (5.58 kb) is obtained. As shown in FIG. 1, synthetic pdc pyruvate decarboxylase gene is inserted in the downstream location of the promoter psbAII, where is the original regions for aphX and sacB gene. Spontaneous transformation is used to insert the expression vector into the Synechocystis mutant S. strictus. The synthetic pdc gene is then integrated into the S. strictus chromosome by means of homologous recombination. After the conversion, the S. strictus mutant cells are grown on Millipore film covering BG-11 solid medium without ampicillin (Amp) for 20 hours, and then Millipore film is transferred to the top of BG-11 solid medium containing 15 μg/ml ampicillin for screening culture. A week later, single green colonies appear on the Millipore film, which are anti-Amp S. Strictus mutants. Repeat the screening cultures on BG-11 solid culture medium and gradually increase the concentration of ampicillin, genetically stable traits of S. strictus mutants are obtained.

In order to verify that pdc gene have been integrated into S. strictus chromosome, and thus the entire path of the ethanol synthesis (pyruvate→acetaldehyde→ethanol) have been connected, single colonies are analyzed on aldehyde indicator plates to verify the activity of the alcohol dehydrogenase enzyme. These indicator plates are formulated by the addition of 8 ml of pararosaniline (2.5 mg of the dry powder/ml of 95% ethanol; not autoclaved) and 100 mg of sodium bisulfite (unsterilized dry powder) to 400-ml batches of LB agar. Mixtures of pararosaniline and bisulfite are often referred to as Schiff reagent. It has been widely used to detect aldehydes, to detect sugars on glycoproteins after periodic acid oxidation, or in a broth to test for organisms which secrete aldehydes into the culture media.

### Example 4

### Copper Induction of Ethanol Production

Standard growth conditions for cyanobacteria in BG-11 liquid and on plates have been described (Buikema, W J; Haselkorn, R. J Bacteriol. 1991; 173:1879-1885). When cyanobacteria strains containing the petE promoter are being constructed, a modified BG-11 medium without copper sulfate is used. Acid-washed, dry-heat-sterilized glassware or disposable plasticware are also used.

Cells are induced with copper by washing exponentially growing cells with fresh BG-11 medium, BG-11 containing no fixed nitrogen (BG-11₀), or BG-11 with 1 mM ammonium sulfate. Specified concentrations of total copper are attained by adding dissolved copper sulfate as needed. For liquid cultures, cells are grown in flasks with shaking at 150 rpm under continuous illumination at 32° C. for 2 days. For slides, 10 μl of a dense cell suspension is placed in the center of a 300-μl 1% (wt/vol) agarose slab containing the appropriate medium, copper, and 10 mM potassium bicarbonate, and covered with a coverslip. Slides are incubated in a clear humid chamber under the same light conditions as the liquid cultures.

### Example 5

### Ethanol Concentration Assay

This Example illustrates determination of the ethanol concentration in a liquid culture. For determination of ethanol concentration of a liquid culture, a 550 μl aliquot of the culture is taken, spun down at 12,100×g for 5 min, and 500 μl (or other appropriate vol.) of the supernatant is placed in a fresh 1.5 ml rube and stored at −20° C. until performing the assay. Given the linear range of the spectrophotometer and the sensitivity of the ethanol assay, dilution of the sample (up to 20 fold) is occasionally required. In this case, an appropriate volume of BG-1 1 is first added to the fresh 1.5 ml tube, to which the required vol. of clarified supernatant is added. This solution is used directly in the ethanol assay. Upon removal from −20° C. and immediately before performing the assay, the samples are spun down a second time at 12,100×g for 5 mm, also assisting in sample thawing.

The Boehringer Mannheim/r-Biopharm® enzymatic ethanol detection kit is used for ethanol concentration determination. Briefly, this assay exploits the action of alcohol dehydrogenase (ADH) and acetaldehyde dehydrogenase in a phosphate-buffered solution of the NAD⁺ cofactor, which upon the addition of ethanol causes a conversion of NAD⁺ to NADH. Concentration of NADH is determined by light absorbance at 340 ran (A34₀) and is then used to determine ethanol concentration. The assay is performed as given in the instructions, with the following modifications. As given under point 4 on the instruction sheet, the maximal sample volume (v=0.5 ml), for maximum sensitivity, is used for the assay. Finally, all volumes in the assay (including the above v=0.5 ml) are quartered. This allows for reagent conservation, and the ability to retain a majority of the sample aliquot's volume, in case repetition is required. Thus, the sample volume used is actually v=0.125 ml, in 0.75 ml of reaction mixture 2, and with the later addition of 12.5 μl of (ADH) suspension 3. This conserved ratio volumetric reduction is determined to have no effect on the assay as performed. BG-1 1 is used as a blank.

### Example 6

### Laboratory Photobioreactor Fermentation

A 1 L indoor photobioreactor modified with CelliGen® Plus (New Brunswick Scientific Inc., Edison, N.J., USA) is used to characterize the S. strictus mutants. The system possesses built-in temperature, pH, speed, control and measurement of dissolved oxygen, and so on. Based on this, adjustable light source is installed so that the reactor wall can be illuminated by the lighting intensity up to 1000 Einstein/m2/s. The Synechocystis cultivation process is monitored and controlled automatically by a Pentium II (233 MHz, Windows 98) computer equipped with an interface board PCI-MIO-16E-10 (National Instruments Corp., Austin, Tex.). The data acquisition program is written in LabVIEW7.1 (National Instruments Corp., Austin, Tex.). In this system, the copper sulfate is used to induce the synthesis of ethanol when the cell density reaches a certain level, for example, 1 gram dry cell weight/liter of medium. As a result, the ethanol concentration in the S. strictus cell cultures is measured to be 20 mM after 5 days of fermentation.

### Example 7

### Outdoor Photobioreactor Fermentation

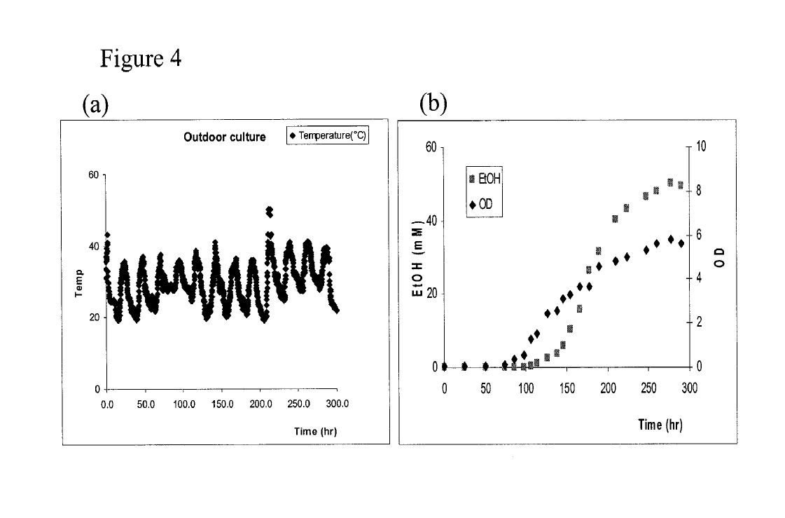

A 10 L outdoor photobioreactor is used for implementation of the suspension culture for ethanol-producing S. strictus mutants. pH control is used to manipulate the amount of carbon dioxide entering the photobioreactor. Temperature is not controlled. A temperature profile of this outdoor photobioreactor system is depicted in FIG. 4a. The air-lift photobioreactor is made by the glass tube with inner circulation device which can be effective in promoting the spread and improve the two-phase gas-liquid mixture to strengthen the process of transfer of carbon dioxide. Synthesis of ethanol is induced by addition of copper sulfate when the cell density reaches a certain level, for example, 1 gram dry cell weight/liter of medium. The ethanol concentration in the S. strictus cell cultures is measured to be approximately 50 mM after about 8 days of fermentation (FIG. 4b).

| Sequence Listing | |
|---|---|
| SEQ ID NO. 1: petE promoter polynucleotide sequence (420 bp): | |
| atgaaattgattgcggcaagcttgcgacgcttaagtttagctgtgttaactgttctttta | |
| gttgttagcagctttgctgtgttcacaccttctgcatcggetgaaacatacacagtaaaa | |
| ctaggtagcgataaaggactgttagtatttgaaccagcaaaattaacaatcaagccaggt | |
| gacacggttgaatttttaaacaacaaagttcctccccataatgttgtgtttgatgctgct | |
| ctaaacccggctaagagtgctgatttagctaagtctttatctcacaaacagttgttaatg | |
| agtcctggccaaagcaccagcactactttcccagcagatgcacccgcaggtgagtacacc | |
| ttctactgcgaacctcaccgtggtgctggtatggttggtaaaatcactgtcgccggctag | |
| SEQ ID NO. 2: petE promoter amino acid (AA) sequence (139 AA) | |
| MKLIAASLRRLSLAVLTVLLVVSSFAVFTPSASAETYTVKLGSDKGLLVFEPAKLTIKPG | |
| DTVEFLNNKVPPHNVVFDAALNPAKSADLAKSLSHKQLLMSPGQSTSTTFPADAPAGEYTF | |
| YCEPHRGAGMVGKITVAG | |
| SEQ ID NO. 3: Pyruvate decarboxylase, pdc, | |
| polynucleotide sequence (1707 bp) | |
| ATGAGTTATACTGTCGGTACCTATTTAGCGGAGCGGCTTGTCCAGATTGGTCTCAAGCA | |
| TCACTTCGCAGTCGCGGGCGACTACAACCTCGTCCTTCTTGACAACCTGCTTTTGAACA | |
| AAAACATGGAGCAGGTTTATTGCTGTAACGAACTGAACTGCGGTTTCAGTGCAGAAGG | |
| TTATGCTCGTGCCAAAGGCGCAGCAGCAGCCGTCGTTACCTACAGCGTCGGTGCGCTTT | |
| CCGCATTTGATGCTATCGGTGGCGCCTATGCAGAAAACCTTCCGGTTATCCTGATCTCC | |
| GGTGCTCCGAACAACAATGATCACGCTGCTGGTCACGTGTTGCATCACGCTCTTGGCAA | |
| AACCGACTATCACTATCAGTTGGAAATGGCCAAGAACATCACGGCCGCAGCTGAAGCG | |
| ATTTACACCCCAGAAGAAGCTCCGGCTAAAATCGATCACGTGATTAAAACTGCTCTTCG | |
| TGAGAAGAAGCCGGTTTATCTCGAAATCGCTTGCAACATTGCTTCCATGCCCTGCGCCG | |
| CTCCTGGACCGGCAAGCGCATTGTTCAATGACGAAGCCAGCGACGAAGCTTCTTTGAAT | |
| GCAGCGGTTGAAGAAACCCTGAAATTCATCGCCAACCGCGACAAAGTTGCCGTCCTCG | |
| TCGGCAGCAAGCTGCGCGCAGCTGGTGCTGAAGAAGCTGCTGTCAAATTTGCTGATGCT | |
| CTCGGTGGCGCAGTTGCTACCATGGCTGCTGCAAAAAGCTTCTTCCCAGAAGAAAACCC | |
| GCATTACATCGGTACCTCATGGGGTGAAGTCAGCTATCCGGGCGTTGAAAAGACGATG | |
| AAAGAAGCCGATGCGGTTATCGCTCTGGCTCCTGTCTTCAACGACTACTCCACCACTGG | |
| TTGGACGGATATTCCTGATCCTAAGAAACTGGTTCTCGCTGAACCGCGTTCTGTCGTCG | |
| TTAACGGCGTTCGCTTCCCCAGCGTTCATCTGAAAGACTATCTGACCCGTTTGGCTCAG | |
| AAAGTTTCCAAGAAAACCGGTGCTTTGGACTTCTTCAAATCCCTCAATGCAGGTGAACT | |
| GAAGAAAGCCGCTCCGGCTGATCCGAGTGCTCCGTTGGTCAACGCAGAAATCGCCCGT | |
| CAGGTCGAAGCTCTTCTGACCCCGAACACGACGGTTATTGCTGAAACCGGTGACTCTTG | |
| GTTCAATGCTCAGCGCATGAAGCTCCCGAACGGTGCTCGCGTTGAATATGAAATGCAGT | |
| GGGGTCACATCGGTTGGTCCGTTCCTGCCGCCTTCGGTTATGCCGTCGGTGCTCCGGAA | |
| CGTCGCAACATCCTCATGGTTGGTGATGGTTCCTTCCAGCTGACGGCTCAGGAAGTCGC | |
| TCAGATGGTTCGCCTGAAACTGCCGGTTATCATCTTCTTGATCAATAACTATGGTTACAC | |
| CATCGAAGTTATGATCCATGATGGTCCGTACAACAACATCAAGAACTGGGATTATGCCG | |
| GTCTGATGGAAGTGTTCAACGGTAACGGTGGTTATGACAGCGGTGCTGGTAAAGGCCT | |
| GAAGGCTAAAACCGGTGGCGAACTGGCAGAAGCTATCAAGGTTGCTCTGGCAAACACC | |
| GACGGCCCAACCCTGATCGAATGCTTCATCGGTCGTGAAGACTGCACTGAAGAATTGGT | |
| CAAATGGGGTAAGCGCGTTGCTGCCGCCAACAGCCGTAAGCCTGTTAACAAGCTCCTCT | |
| AGTTTTTGGGGATCAATTCGAGCTCGGTACCCAAACTAGTATGTAGGGTGAGGTTATAG | |
| CT | |
| SEQ ID NO. 4: PCR primer P1 for pdc gene | |
| P1 (5-end primer): 5′-GGAGTAAGCATATGAGTTATACTG- 3′ | |
| SEQ ID NO. 5: PCR primer P2 for pdc gene: | |
| P2 (3′end primer): 5′- GGATCTCGACTCTAGAGGATCC- 3′ | |
| SEQ ID NO. 6: pPETPDC polynucleotide sequence | |
| 1 | TCTTCCGCTTCCTCGCTCACTGACTCGCTGCGCTCGGTCGTTCGGCTGCGGCGAGCGGTA |
| 61 | TCAGCTCACTCAAAGGCGGTAATACGGTTATCCACAGAATCAGGGGATAACGCAGGAAAG |
| 121 | AACATGTGAGCAAAAGGCCAGCAAAAGGCCAGGAACCGTAAAAAGGCCGCGTTGCTGGCG |
| 181 | TTTTTCCATAGGCTCCGCCCCCCTGACGAGCATCACAAAAATCGACGCTCAAGTCAGAGG |
| 241 | TGGCGAAACCCGACAGGACTATAAAGATACCAGGCGTTTCCCCCTGGAAGCTCCCTCGTG |
| 301 | CGCTCTCCTGTTCCGACCCTGCCGCTTACCGGATACCTGTCCGCCTTTCTCCCTTCGGGA |
| 361 | AGCGTGGCGCTTTCTCATAGCTCACGCTGTAGGTATCTCAGTTCGGTGTAGGTCGTTCGC |
| 421 | TCCAAGCTGGGCTGTGTGCACGAACCCCCCGTTCAGCCCGACCGCTGCGCCTTATCCGGT |
| 481 | AACTATCGTCTTGAGTCCAACCCGGTAAGACACGACTTATCGCCACTGGCAGCAGCCACT |
| 541 | GGTAACAGGATTAGCAGAGCGAGGTATGTAGGCGGTGCTACAGAGTTCTTGAAGTGGTGG |
| 601 | CCTAACTACGGCTACACTAGAAGAACAGTATTTGGTATCTGCGCTCTGCTGAAGCCAGTT |
| 661 | ACCTTCGGAAAAAGAGTTGGTAGCTCTTGATCCGGCAAACAAACCACCGCTGGTAGCGGT |
| 721 | GGTTTTTTTGTTTGCAAGCAGCAGATTACGCGCAGAAAAAAAGGATCTCAAGAAGATCCT |
| 781 | TTGATCTTTTCTACGGGGTCTGACGCTCAGTGGAACGAAAACTCACGTTAAGGGATTTTG |
| 841 | GTCATGAGATTATCAAAAAGGATCTTCACCTAGATCCTTTTAAATTAAAAATGAAGTTTT |
| 901 | AAATCAATCTAAAGTATATATGAGTAAACTTGGTCTGACAGTTACCAATGCTTAATCAGT |
| 961 | GAGGCACCTATCTCAGCGATCTGTCTATTTCGTTCATCCATAGTTGCCTGACTCCCCGTC |
| 1021 | GTGTAGATAACTACGATACGGGAGGGCTTACCATCTGGCCCCAGTGCTGCAATGATACCG |
| 1081 | CGAGACCCACGCTCACCGGCTCCAGATTTATCAGCAATAAACCAGCCAGCCGGAAGGGCC |
| 1141 | GAGCGCAGAAGTGGTCCTGCAACTTTATCCGCCTCCATCCAGTCTATTAATTGTTGCCGG |
| 1201 | GAAGCTAGAGTAAGTAGTTCGCCAGTTAATAGTTTGCGCAACGTTGTTGCCATTGCTACA |
| 1261 | GGCATCGTGGTGTCACGCTCGTCGTTTGGTATGGCTTCATTCAGCTCCGGTTCCCAACGA |
| 1321 | TCAAGGCGAGTTACATGATCCCCCATGTTGTGCAAAAAAGCGGTTAGCTCCTTCGGTCCT |
| 1381 | CCGATCGTTGTCAGAAGTAAGTTGGCCGCAGTGTTATCACTCATGGTTATGGCAGCACTG |
| 1441 | CATAATTCTCTTACTGTCATGCCATCCGTAAGATGCTTTTCTGTGACTGGTGAGTACTCA |
| 1501 | ACCAAGTCATTCTGAGAATAGTGTATGCGGCGACCGAGTTGCTCTTGCCCGGCGTCAATA |
| 1561 | CGGGATAATACCGCGCCACATAGCAGAACTTTAAAAGTGCTCATCATTGGAAAACGTTCT |
| 1621 | TCGGGGCGAAAACTCTCAAGGATCTTACCGCTGTTGAGATCCAGTTCGATGTAACCCACT |
| 1681 | CGTGCACCCAACTGATCTTCAGCATCTTTTACTTTCACCAGCGTTTCTGGGTGAGCAAAA |
| 1741 | ACAGGAAGGCAAAATGCCGCAAAAAAGGGAATAAGGGCGACACGGAAATGTTGAATACTC |
| 1801 | ATACTCTTCCTTTTTCAATATTATTGAAGCATTTATCAGGGTTATTGTCTCATGAGCGGA |
| 1861 | TACATATTTGAATGTATTTAGAAAAATAAACAAATAGGGGTTCCGCGCACATTTCCCCGA |
| 1921 | AAAGTGCCACCTGACGTCTAAGAAACCATTATTATCATGACATTAACCTATAAAAATAGG |
| 1981 | CGTATCACGAGGCCCTTTCGTCTCGCGCGTTTCGGTGATGACGGTGAAAACCTCTGACAC |
| 2041 | ATGCAGCTCCCGGAGACGGTCACAGCTTGTCTGTAAGCGGATGCCGGGAGCAGACAAGCC |
| 2101 | CGTCAGGGCGCGTCAGCGGGTGTTGGCGGGTGTCGGGGCTGGCTTAACTATGCGGCATCA |
| 2161 | GAGCAGATTGTACTGAGAGTGCACCATAAAATTGTAAACGTTAATATTTTGTTAAAATTC |
| 2221 | GCGTTAAATTTTTGTTAAATCAGCTCATTTTTTAACCAATAGGCCGAAATCGGCAAAATC |
| 2281 | CCTTATAAATCAAAAGAATAGCCCGAGATAGGGTTGAGTGTTGTTCCAGTTTGGAACAAG |
| 2341 | AGTCCACTATTAAAGAACGTGGACTCCAACGTCAAAGGGCGAAAAACCGTCTATCAGGGC |
| 2401 | GATGGCCCACTACGTGAACCATCACCCAAATCAAGTTTTTTGGGGTCGAGGTGCCGTAAA |
| 2461 | GCACTAAATCGGAACCCTAAAGGGAGCCCCCGATTTAGAGCTTGACGGGGAAAGCCGGCG |
| 2521 | AACGTGGCGAGAAAGGAAGGGAAGAAAGCGAAAGGAGCGGGCGCTAGGGCGCTGGCAAGT |
| 2581 | GTAGCGGTCACGCTGCGCGTAACCACCACACCCGCCGCGCTTAATGCGCCGCTACAGGGC |
| 2641 | GCGTACTATGGTTGCTTTGACGTATGCGGTGTGAAATACCGCACAGATGCGTAAGGAGAA |
| 2701 | AATACCGCATCAGGCGCCATTCGCCATTCAGGCTGCGCAACTGTTGGGAAGGGCGATCGG |
| 2761 | TGCGGGCCTCTTCGCTATTACGCCAGCTGGCGAAAGGGGGATGTGCTGCAAGGCGATTAA |
| 2821 | GTTGGGTAACGCCAGGGTTTTCCCAGTCACGACGTTGTAAAACGACGGCCAGTGCCAAGC |
| 2881 | TTAAGGTGCACGGCCCACGTGGCCACTAGTACTTCTCGAGCTCTGTACATGTCCGCGGTC |
| 2941 | GCGACGTACGCGTATCGATGGCGCCAGGAGAGAGAGCTGCAGAGCGTTCCAGTGGATATT |
| 3001 | TGCTGGGGGTTAATGAAACATTGTGGCGGAACCCAGGGACAATGTGACCAAAAAATTCAG |
| 3061 | GGATATCAATAAGTATTAGGTATATGGATCATAATTGTATGCCCGACTATTGCTTAAACT |
| 3121 | GACTGACCACTGACCTTAAGAGTAATGGCGTGCAAGGCCCAGTGATCAATTTCATTATTT |
| 3181 | TTCATTATTTCATCTCCATTGTCCCTGAAAATCAGTTGTGTCGCCCCTCTACACAGCCCA |
| 3241 | GAACTATGGTAAAGGCGCACGAAAAACCGCCAGGTAAACTCTTCTCAACCCCCAAAACGC |
| 3301 | CCTCTGTTTACCCATTCCCTCTCAGCTCAAAAAGTATCAATGATTACTTAATGTTTGTTC |
| 3361 | TGCGCAAACTTCTTGCAGAACATGCATGATTTACAAAAAGTTGTAGTTTCTGTTACCAAT |
| 3421 | TGCGAATCGAGAACTGCCTAATCTGCCGAGTATGCAAGCTGCTTTGTAGGCAGATGAACA |
| 3481 | TATGATATATATAATTAGCGGAGCGGCTTGTCCAGATTGGTCTCAAGCATCACTTCGCAG |
| 3541 | CGCGGGCGACTACAACCTCGTCCTTCTTGACAACCTGCTTTTGAACAAAAACATGGAGCA |
| 3601 | GGTTTATTGCTGTAACGAACTGAACTGCGGTTTCAGTGCAGAAGGTTATGCTCGTGCCAA |
| 3661 | AGGCGCAGCAGCAGCCGTCGTTACCTACAGCGTCGGTGCGCTTTCCGCATTTGATGCTAT |
| 3721 | CGGTGGCGCCTATGCAGAAAACCTTCCGGTTATCCTGATCTCCGGTGCTCCGAACAACAA |
| 3781 | TGATCACGCTGCTGGTCACGTGTTGCATCACGCTCTTGGCAAAACCGACTATCACTATCA |
| 3841 | GTTGGAAATGGCCAAGAACATCACGGCCGCAGCTGAAGCGATTTACACCCCAGAAGAAGC |
| 3901 | TCCGGCTAAAATCGATCACGTGATTAAAACTGCTCTTCGTGAGAAGAAGCCGGTTTATCT |
| 3961 | CGAAATCGCTTGCAACATTGCTTCCATGCCCTGCGCCGCTCCTGGACCGGCAAGCGCATT |
| 4021 | GTTCAATGACGAAGCCAGCGACGAAGCTTCTTTGAATGCAGCGGTTGAAGAAACCCTGAA |
| 4081 | ATTCATCGCCAACCGCGACAAAGTTGCCGTCCTCGTCGGCAGCAAGCTGCGCGCAGCTGG |
| 4141 | TGCTGAAGAAGCTGCTGTCAAATTTGCTGATGCTCTCGGTGGCGCAGTTGCTACCATGGC |
| 4201 | TGCTGCAAAAAGCTTCTTCCCAGAAGAAAACCCGCATTACATCGGTACCTCATGGGGTGA |
| 4261 | AGTCAGCTATCCGGGCGTTGAAAAGACGATGAAAGAAGCCGATGCGGTTATCGCTCTGGC |
| 4321 | TCCTGTCTTCAACGACTACTCCACCACTGGTTGGACGGATATTCCTGATCCTAAGAAACT |
| 4381 | GGTTCTCGCTGAACCGCGTTCTGTCGTCGTTAACGGCGTTCGCTTCCCCAGCGTTCATCT |
| 4441 | GAAAGACTATCTGACCCGTTTGGCTCAGAAAGTTTCCAAGAAAACCGGTGCTTTGGACTT |
| 4501 | CTTCAAATCCCTCAATGCAGGTGAACTGAAGAAAGCCGCTCCGGCTGATCCGAGTGCTCC |
| 4561 | GTTGGTCAACGCAGAAATCGCCCGTCAGGTCGAAGCTCTTCTGACCCCGAACACGACGGT |
| 4621 | TATTGCTGAAACCGGTGACTCTTGGTTCAATGCTCAGCGCATGAAGCTCCCGAACGGTGC |
| 4681 | TCGCGTTGAATATGAAATGCAGTGGGGTCACATCGGTTGGTCCGTTCCTGCCGCCTTCGG |
| 4741 | TTATGCCGTCGGTGCTCCGGAACGTCGCAACATCCTCATGGTTGGTGATGGTTCCTTCCA |
| 4801 | GCTGACGGCTCAGGAAGTCGCTCAGATGGTTCGCCTGAAACTGCCGGTTATCATCTTCTT |
| 4861 | GATCAATAACTATGGTTACACCATCGAAGTTATGATCCATGATGGTCCGTACAACAACAT |
| 4921 | CAAGAACTGGGATTATGCCGGTCTGATGGAAGTGTTCAACGGTAACGGTGGTTATGACAG |
| 4981 | CGGTGCTGGTAAAGGCCTGAAGGCTAAAACCGGTGGCGAACTGGCAGAAGCTATCAAGGT |
| 5041 | TGCTCTGGCAAACACCGACGGCCCAACCCTGATCGAATGCTTCATCGGTCGTGAAGACTG |
| 5101 | CACTGAAGAATTGGTCAAATGGGGTAAGCGCGTTGCTGCCGCCAACAGCCGTAAGCCTGT |
| 5161 | TAACAAGCTCCTCTAGTTTTTGGGGATCAATTCGAGCTCGGTACCCAAACTAGTATGTAG |
| 5221 | GGTGAGGTTATAGCTTAATTCCTTGGTGTAATGCCAACTGAATAATCTGCAAATTGCACT |
| 5281 | CTCCTTCAATGGGGGGTGCTTTTTGCTTGACTGAGTAATCTTCTGATTGCTGATCTTGAT |
| 5341 | TGCCATCGATCGCCGGGGAGTCCGGGGCAGTTACCATTAGAGAGTCTAGAGAATTAATCC |
| 5401 | ATCTTCGATAGAGGAATTATGGGGGAAGAACCTGTGCCGGCGGATAAAGCATTAGGCAAG |
| 5461 | AAATTCAAGAAAAAAAATGCCTCCTGGAGCATTGAAGAAAGCGAAGCTCTGTACCGGGTT |
| 5521 | GAGGCCTGGGGGGCACCTTATTTTGCCATTAATGCCGCTGGTAACATAACCGTCTCTCCC |
| 5581 | AACGGCGATCGGGGCGGTTCGTTAGATTTGTTGGAACTGGTGGAAGCCCTGCGGCAAAGA |
| 5641 | AAGCTCGGCTTACCCCTATTAATTCGTTTTTCCGATATTTTGGCCGATCGCCTAGAGCGA |
| 5701 | TTGAATAGTTGTTTTGCCAAGGCGATCGAATTCGTAATCATGGTCATAGCTGTTTCCTGT |
| 5761 | GTGAAATTGTTATCCGCTCACAATTCCACACAACATACGAGCCGGAAGCATAAAGTGTAA |
| 5821 | AGCCTGGGGTGCCTAATGAGTGAGCTAACTCACATTAATTGCGTTGCGCTCACTGCCCGC |
| 5881 | TTTCCAGTCGGGAAACCTGTCGTGCCAGCTGCATTAATGAATCGGCCAACGCGCGGGGAG |
| 5941 | AGGCGGTTTGCGTATTGGGCGC |

SEQ ID NO. 8
Pyruvate decarboxylase, pdc, amino acid (AA) sequence (568 AA)
MSYTVGTYLAERLVQIGLKHHFAVAGDYNLVLLDNLLLNKNMEQVYCCNELNCGESAEG
YARAKGAAAAVVTYSVGALSAFDAIGGAYAENLPVILISGAPNNNDHAAGHVLHHALGKT
DYHYQLEMAKNITAAAEAIYTPEEAPAKIDHVIKTALREKKPVYLEIACNIASMPCAAPGP
ASALFNDEASDEASLNAAVEETLKFIANRDKVAVLVGSKLRAAGAEEAAVKFADALGGAV
ATMAAAKSFFPEENPHYIGTSWGEVSYPGVEKTMKEADAVIALAPVFNDYSTTGWTDIPD
PKKLVLAEPRSVVVNGIRFPSVHLKDYLTRLAQKVSKKTGALDFFKSLNAGELKKAAPAD
PSAPLVNAEIARQVEALLTPNTTVIAETGDSWFNAQRMKLPNGARVEYEMQWGHIGWSVP
AAFGYAVGAPERRNILMVGDGSFQLTAQEVAQMVRLKLPVIIFLINNYGYTIEVMIHDGP
YNNIKNWDYAGLMEVFNGNGGYDSGAGKGLKAKTGGELAEAIKVALANTDGPTLIECFIG
REDCTEELVKWGKRVAAANSRKPVNKLL

What is claimed is:

1. A polynucleotide construct comprising: a copper ion inducible promoter and a polynucleotide sequence encoding a pyruvate decarboxylase (pdc) enzyme.

2. The nucleic acid construct of claim 1, wherein the copper ion inductive promoter is pPetE promoter.

3. The nucleic acid construct of claim 1, wherein the polynucleotide sequence encoding pdc enzyme is obtained from Acetobacter pasteurianus plasmid pGADL201.

4. The nucleic acid construct of claim 1, wherein the polynucleotide sequence encoding pdc enzyme is obtained from Gluconobacter suboxydans.

5. The nucleic acid construct of claim 1, wherein the polynucleotide sequence encoding pdc enzyme comprises SEQ. ID NO: 3 or a pdc enzyme-encoding polynucleotide sequence that is capable of being expressed in cyanobacteria.

6. The nucleic acid construct of claim 1, wherein the sequence encoding pdc enzyme comprises a nucleic acid sequence encoding an amino acid sequence of SEQ ID NO: 8.

7. An expression vector comprising a polynucleotide construct of claim 1.

8. A host cell comprising the expression vector of claim 7.

9. The host cell of claim 8, wherein the expression vector is integrated into the host cell chromosome.

10. The host cell of claim 8, wherein the expression vector is pPETPDC.

11. The host cell of claim 8, wherein the cell is a cyanobacterium.

12. The host cell of claim 11, wherein the cyanobacterium is Synechocystis.

13. The host cell of claim 11, wherein the cyanobacterium is Synechocystis sp. PCC 6803, or other transformable strain of Synechocystis.

14. The host cell of claim 11, wherein the cyanobacterium is a wild-type strain of Synechocystis sp. PCC 6803.

15. The host cell of claim 11, wherein the cyanobacterium is Synechococcus PCC 7942, or other transformable strain of Synechococcus.

16. The host cell of claim 11, wherein the host cell produces ethanol in a quantifiable amount after a period of copper ion induction.

17. The host cell of claim 11, wherein the host cell produces ethanol in a quantity that is greater than about 50 mM ethanol after about 8 days of fermentation.

18. A genetically engineered cyanobacterium comprising a polynucleotide construct, which comprises a polynucleotide sequence encoding pyruvate decarboxylase (pdc) enzyme and a copper ion inducible promoter, wherein the cyanobacterium is capable of producing ethanol.

19. The cyanobacterium of claim 18, wherein the ethanol is produced in a quantity that is greater than about 50 mM after about 8 days of fermentation.

20. The cyanobacterium of claim 18, wherein the cyanobacterium is resistant to high temperature and high ethanol concentration.

21-41. (canceled)
